(19) United States
(12) Patent Application Publication
(10) Pub. No.: US 2022/0157460 A1
(43) Pub. Date: May 19, 2022

(54) METHOD AND DEVICE FOR ANALYZING RESULT FROM SELF-DIAGNOSIS APPARATUS

(71) Applicant: SMILELAB CO., LTD., Seoul (KR)

(72) Inventors: Kyou Hee LEE, Yongin-si (KR); Young Hun SONG, Anyang-si (KR)

(73) Assignee: SMILELAB CO., LTD., Seoul (KR)

(21) Appl. No.: 17/429,535

(22) PCT Filed: Jan. 29, 2020
(86) PCT No.: PCT/KR2020/001323

§ 371 Date: Aug. 9, 2021

(30) Foreign Application Priority Data
Feb. 12, 2019 (KR) 10-2019-0016288

(51) Int. Cl. G16H 50/20; G06T 7/00

(52) U.S. Cl. CPC G16H 50/20; A61B 10/0012; G06T 2207/30004; G06T 7/0012

(57) ABSTRACT

Proposed herein are a method and device for analyzing a diagnosis result of a self-diagnosis apparatus. The device for analyzing a diagnosis result of a self-diagnosis apparatus includes: memory configured to store guide information, which is information about a method of using a self-diagnosis apparatus; and a control unit configured to provide the guide information for the self-diagnosis apparatus selected by a user, and to analyze a diagnosis result of the self-diagnosis apparatus tested according to the provided guide information.

### TECHNICAL FIELD

The embodiments disclosed herein relate to a method and device for analyzing the result of a self-diagnosis apparatus, and more particularly to a method and device for analyzing the result of a self-diagnosis apparatus that provide an accurate diagnosis result by analyzing the diagnosis result of a self-diagnosis apparatus while providing a user guide for each self-diagnosis apparatus used by a user.

### BACKGROUND ART

Recently, as self-diagnosis apparatuses having various purposes have been developed, users can be easily checked for their physical conditions.

To this end, each self-diagnosis apparatus reacts with a specific hormone or protein, and changes its color or displays a specific mark, thereby diagnosing a specific disease or physical condition.

In particular, in the case of self-diagnosis apparatuses using a hormone, for example, for a pregnancy test or an ovulation check, a reagent that reacts with a hormone appearing during pregnancy or ovulation is applied to each of the self-diagnosis apparatuses. As the reagent reacts with the hormone, a user can check whether she is pregnant or ovulating depending on whether the color of the reagent applied to the self-diagnosis apparatus changes.

In this case, as the time for the reaction with the reagent and a detection method vary depending on the hormone or protein to be detected for each self-diagnosis apparatus, a sampling method, a usage method, and the reading of a result vary for each self-diagnosis apparatus. Even for the same type of diagnosis apparatus, the usage method varies depending on the manufacturer or model.

However, since the shapes and operating principles of the self-diagnosis apparatuses are similar, a problem arises in that a user may obtain an incorrect diagnosis result because the user does not know exactly how to use it or how to read a result.

In connection with this, Korea Patent Application Publication No. 10-2013-0025463, which is a related art document, is directed to a self-diagnosis kit that can easily check the health condition of the body. A module unit consisting of a blood glucose measurement module and an activated oxygen measurement module to the main body of the meter is selectively attached and used so that the self-diagnosis kit is easy to carry and can be used easily at home. Only terminals are individually formed on individual modules and the main body of the measuring instrument according to the difference between the signal obtained during blood glucose measurement and the signal obtained from acceleration pulse waves and heart rate variability. This technology cannot induce a user to use the self-diagnosis apparatus correctly or provide accurate result reading.

Therefore, there is a need for a technology for overcoming the above-described problems.

Meanwhile, the above-described background technology corresponds to technical information that has been possessed by the present inventor in order to contrive the present invention or that has been acquired in the process of contriving the present invention, and can not necessarily be regarded as well-known technology that had been known to the public prior to the filing of the present invention.

### DISCLOSURE

### Technical Problem

An object of the embodiments disclosed herein is to propose a method and device for analyzing the result of a self-diagnosis apparatus.

An object of the embodiments disclosed herein is to propose a method and device for analyzing the result of a self-diagnosis apparatus that provide an accurate method of using the self-diagnosis apparatus.

An object of the embodiments disclosed herein is to propose a method and device for analyzing the result of a self-diagnosis apparatus that provide an accurate diagnosis result by analyzing the diagnosis result of a self-diagnosis apparatus in which a specimen is sampled.

An object of the embodiments disclosed herein is to propose a method and device for analyzing the result of a self-diagnosis apparatus that determine the reliability of a diagnosis result by comparing and analyzing the diagnosis result of another user group similar to a user and using a self-diagnosis apparatus and the diagnosis result of the user.

An object of the embodiments disclosed herein is to propose a method and device for analyzing the result of a self-diagnosis apparatus that increase the accuracy of a diagnosis result through additional diagnosis by notifying a user of the diagnosis time for additional diagnosis.

### Technical Solution

As a technical solution for accomplishing the above objects, according to an embodiment, there is provided a device for analyzing a diagnosis result of a self-diagnosis apparatus, the device including: memory configured to store guide information, which is information about a method of using a self-diagnosis apparatus; and a control unit configured to provide the guide information for the self-diagnosis apparatus selected by a user, and to analyze a diagnosis result of the self-diagnosis apparatus tested according to the provided guide information.

According to another embodiment, there is provided a method by which a device for analyzing a self-diagnosis apparatus analyzes a diagnosis result of a self-diagnosis apparatus, the method including: providing guide information, which is information about a method of using a self-diagnosis apparatus selected by a user; and analyzing a diagnosis result of the self-diagnosis apparatus tested according to the provided guide information.

According to still another embodiment, there is provided a computer-readable storage medium having stored thereon a program that performs a method for analyzing a self-diagnosis apparatus, wherein the method of analyzing a self-diagnosis apparatus includes providing guide information, which is information about a method of using a self-diagnosis apparatus selected by a user, and analyzing a diagnosis result of the self-diagnosis apparatus tested according to the provided guide information.

According to another embodiment, there is provided a computer program that is executed by a device for analyzing a self-diagnosis apparatus and stored in a storage medium in order to a method of analyzing a self-diagnosis apparatus, wherein the method of analyzing a self-diagnosis apparatus includes providing guide information, which is information about a method of using a self-diagnosis apparatus selected by a user, and analyzing a diagnosis result of the self-diagnosis apparatus tested according to the provided guide information.

### Advantageous Effects

According to any one of the above-described technical solutions, there may be proposed the method and device for analyzing the result of a self-diagnosis apparatus.

According to any one of the above-described technical solutions, there may be proposed the method and device for analyzing the result of a self-diagnosis apparatus that allow a user to accurately use a self-diagnosis apparatus through guidance on an accurate method of using the self-diagnosis apparatus.

According to any one of the above-described technical solutions, there may be proposed the method and device for analyzing the result of a self-diagnosis apparatus that identify an accurate diagnosis result even when a diagnosis result is unclear to the naked eye by An object of the embodiments disclosed herein is to propose a method and device for analyzing the result of a self-diagnosis apparatus that provides an accurate diagnosis result by analyzing the diagnosis result of a self-diagnosis apparatus in which a specimen is sampled.

According to any one of the above-described technical solutions, there may be proposed the method and device for analyzing the result of a self-diagnosis apparatus that determine the reliability of a diagnosis result by comparing and analyzing the diagnosis result of another user group similar to a user using a self-diagnosis apparatus and the diagnosis result of the user.

According to any one of the above-described technical solutions, there may be proposed the method and device for analyzing the result of a self-diagnosis apparatus that increase the accuracy of a diagnosis result through additional diagnosis by notifying a user of the diagnosis time for additional diagnosis.

The effects that can be obtained by the embodiments disclosed herein are not limited to the above-described effects, and other effects that have not been described above will be clearly understood by those of ordinary skill in the art, to which the present invention pertains, from the following description.

### DESCRIPTION OF DRAWINGS

FIG. 1 is a block diagram showing a device for analyzing a self-diagnosis apparatus according to an embodiment;

FIG. 2 is a flowchart illustrating a method of analyzing a self-diagnosis apparatus according to an embodiment; and

FIGS. 3 to 5 are diagrams illustrating a method of analyzing a self-diagnosis apparatus according to an embodiment.

### MODE FOR INVENTION

Various embodiments will be described in detail below with reference to the accompanying drawings. The following embodiments may be modified to various different forms and then practiced. In order to more clearly illustrate features of the embodiments, detailed descriptions of items that are well known to those having ordinary skill in the art to which the following embodiments pertain will be omitted. Furthermore, in the drawings, portions unrelated to descriptions of the embodiments will be omitted.

Throughout the specification, like reference symbols will be assigned to like portions.

Throughout the specification, when one component is described as being “connected” to another component, this includes not only a case where the one component is ‘directly connected’ to the other component but also a case where the one component is ‘connected’ to the other component with a third component arranged therebetween. Furthermore, when one portion is described as “including” one component, this does not mean that the portion does not exclude another component but means that the portion may further include another component, unless explicitly described to the contrary.

The embodiments will be described in detail below with reference to the accompanying drawings.

Prior to the following description, the meanings of the terms to be described below are defined first.

A ‘self-diagnosis apparatus’ is an apparatus that can diagnose a specific disease or a physical condition by the user, and a substance that reacts with a protein or a hormone, which is an index indicating a specific disease or a physical condition, may be applied to the self-diagnosis apparatus.

A ‘specimen’ is a sample taken from a user's body to diagnose a user's disease or physical condition.

Terms that are not defined above will be defined below whenever necessary.

In addition to the terms defined above, terms requiring descriptions will be described separately below.

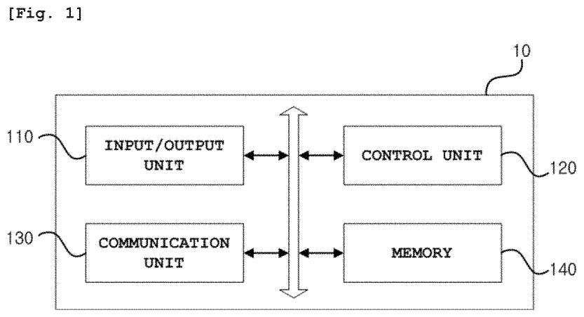

FIG. 1 is a block diagram illustrating a device 10 for analyzing a self-diagnosis apparatus according to an embodiment.

The device 10 for analyzing a self-diagnosis apparatus may be implemented as a computer, a portable terminal, a television, a wearable device, or the like that can access a remote server or can be connected to another terminal and a server over a network N. In this case, the computer includes, e.g., a notebook, a desktop, a laptop, and the like that are each equipped with a web browser. The portable terminal is, e.g., a wireless communication device capable of guaranteeing portability and mobility, and may include all types of handheld wireless communication devices, such as a Personal Communication System (PCS) terminal, a Personal Digital Cellular (PDC) terminal, a Personal Handyphone System (PHS) terminal, a Personal Digital Assistant (PDA), a Global System for Mobile communications (GSM) terminal, an International Mobile Telecommunication (IMT)-2000 terminal, a Code Division Multiple Access (CDMA)-2000 terminal, a W-Code Division Multiple Access (W-CDMA) terminal, a Wireless Broadband (Wibro) Internet terminal, a smartphone, a Mobile Worldwide Interoperability for Microwave Access (mobile WiMAX) terminal, and the like. Furthermore, the television may include an Internet Protocol Television (IPTV), an Internet Television (Internet TV), a terrestrial TV, a cable TV, and the like. Moreover, the wearable device is an information processing device of a type that can be directly worn on a human body, such as a watch, glasses, an accessory, clothing, shoes, or the like, and can access a remote server or be connected to another terminal directly or via another information processing device over a network.

Referring to FIG. 1, the device 10 for analyzing a self-diagnosis apparatus according to the embodiment may include an input/output unit 110, a control unit 120, a communication unit 130, and memory 140.

The input/output unit 110 may include an input unit configured to receive input from a user and an output unit configured to display information about the results of performance of work, the status of the device 10 for analyzing a self-diagnosis apparatus, and the like. For example, input/output unit 110 may include an operation panel configured to receive user input and a display panel configured to display screens.

More specifically, the input unit may include devices capable of receiving various types of user input, such as a keyboard, physical buttons, a touch screen, a camera, a microphone, and the like. Furthermore, the output unit may include a display panel, a speaker, and the like. However, the input/output unit 110 is not limited thereto, but may include various components configured to support various types of input and output.

The input/output unit 110 may photograph the reaction result of the self-diagnosis apparatus through the camera of the input unit, and may provide the result to be photographed to the control unit 120 to be described later.

For example, the input/output unit 110 may photograph a part capable of showing the reaction result of a pregnancy test kit, which is a self-diagnosis apparatus, with a camera.

The control unit 120 controls the overall operation of the device 10 for analyzing a self-diagnosis apparatus, and may include a processor such as a CPU or the like. The control unit 120 may control other components included in the device 10 for analyzing a self-diagnosis apparatus so that they can perform an operation corresponding to user input received via the input/output unit 110.

For example, the control unit 120 may execute a program stored in the memory 140, may read a file stored in the memory 140, or may store a new file in the memory 140.

The control unit 120 may provide guide information that is information about a method of using a self-diagnosis apparatus selected by a user.

To this end, the control unit 120 may receive a selection of a self-diagnosis apparatus to be used by the user. For example, the control unit 120 may scan a barcode printed on the packaging of the self-diagnosis apparatus to be used by the user through the input/output unit 110, and may identify the self-diagnosis apparatus to be used by the user based on the barcode.

Alternatively, for example, the control unit 120 may provide the user with a list including the name of at least one self-diagnosis apparatus, and may receive a selection of any one self-diagnosis apparatus from the provided list from the user.

In addition, the control unit 120 may provide a checklist configured to obtain the user's personal information.

For example, the control unit 120 may generate a checklist including queries on the user's age, a medication being taken, a pre-existing disease, and/or the like in order to obtain the user's physical characteristics, and may obtain the user's personal information by providing the generated checklist to the user.

Alternatively, for example, the control unit 120 may generate a checklist including queries on whether the user has fasted, how to store a self-diagnosis apparatus, whether a self-diagnosis apparatus has been opened, and/or the like, and may check whether the user is ready for an accurate test using the self-diagnosis apparatus through the generated checklist.

In addition, the control unit 120 may analyze the user's physical characteristics or life pattern based on the user's personal information obtained through the provided checklist.

For example, the control unit 120 may analyze the medication being taken by the user and time through the checklist, and may analyze the dosage pattern of the medication, which may affect the reaction result of the self-diagnosis apparatus, based on the raw materials of the medication being taken and the retention time of the medication within the body.

In this case, according to an embodiment, the control unit 120 may classify the user based on the user's personal information, e.g., the user's weight, body mass index, waist circumference, active time, basal body temperature, and/or menstrual date, and/or the like.

For example, the control unit 120 may classify the user by matching the user against other users having a life pattern similar to the user's life pattern analyzed based on the user's personal information. Through this, a plurality of users who use the self-diagnosis apparatus may be grouped into groups of users having similar life patterns.

Thereafter, the control unit 120 may provide guide information about the self-diagnosis apparatus selected by the user.

For example, the control unit 120 may provide guide information about a sampling method such as ‘sampling using flowing urine’ or ‘exposure to a paper cup containing urine for a predetermined period of time’ according to the sampling method of the self-diagnosis apparatus selected by the user.

In this case, according to an embodiment, the control unit 120 may provide guide information based on the user's physical characteristics or life pattern obtained through the checklist.

For example, the control unit 120 may provide guide information adapted to set the specimen collection time for an ovulation test between 10 a.m. and 11 a.m. as the guide information for an ovulation test kit, which is a self-diagnosis apparatus, selected by the user based on the life pattern of the user who is active during the day.

Alternatively, for example, the control unit 120 may provide the available date of the self-diagnosis apparatus by taking into consideration 24 hours, which is the retention time of the medication taken by the user.

In addition, the control unit 120 may request a user input for a test process according to the guide information about the self-diagnosis apparatus, and may check the test process for the self-diagnosis apparatus depending on whether the user input has been obtained.

For example, when it is necessary to bring the ovulation test kit selected by the user into contact with urine for a predetermined period of time in order to collect a specimen, the controller 120 may identify whether the ovulation test kit is used according to the guide information by requesting the input of the start time at which the ovulation test kit is immersed in urine. In addition, the control unit 120 may provide an end alarm when a preset period of time has elapsed from the start time obtained from the user.

In addition, the control unit 120 may analyze the diagnosis result of the tested self-diagnosis apparatus according to the provided guide information.

To this end, the control unit 120 may photograph a reaction state in which the self-diagnosis apparatus reacts with the specimen in response to the test of the user.

For example, the control unit 120 may photograph the pregnancy test kit in which the specimen is sampled using the camera of the input/output unit 110, and may identify a reaction result in which the specimen reacts and changes its color.

In addition, the control unit 120 may determine a diagnosis result based on an image acquired by photographing the self-diagnosis apparatus.

According to an embodiment, the control unit 120 may determine a diagnosis result by analyzing a reaction result corresponding to the self-diagnosis apparatus tested by the user based on a pre-stored reaction result corresponding to the diagnosis result.

For example, the control unit 120 may compare the image acquired by photographing the pregnancy test kit tested by the user based on pre-stored control images acquired by photographing the reaction result of the pregnancy test kit in a pregnancy state and the reaction result of the pregnancy test kit in a non-pregnancy state, and may determine a diagnosis result by calculating the similarity between the image acquired by photographing the pregnancy test kit of the user and the control images.

In this case, according to an embodiment, the control unit 120 may determine a diagnosis result by analyzing the reaction result of the self-diagnosis apparatus of the user based on an image acquired by photographing the reaction result of a self-diagnosis apparatus corresponding to the diagnosis result of another user having a life pattern similar to that of the user.

According to another embodiment, the control unit 120 may learn the relationship between the reaction result of the self-diagnosis apparatus of another user having a life pattern similar to that of the user and a diagnosis result, and may determine a diagnosis result by analyzing the reaction result of the self-diagnosis apparatus of the user based on the result of the learning.

For example, the control unit 120 may learn an image obtained by photographing the reaction result of the pregnancy test kit of another user having a life pattern similar to that of the user according to a pregnancy diagnosis result, and may determine a diagnosis result according to the reaction result of the pregnancy test kit of the user based on the result of the learning.

In addition, the control unit 120 may calculate the reliability, which is a probability indicating whether the diagnosis result determined by analyzing the reaction result of the self-diagnosis apparatus of the user and an actual physical condition are identical to each other.

According to an embodiment, the control unit 120 may provide the reliability of the diagnosis result to the user based on the input of another user having physical characteristics identical or similar to those of the user.

For example, the control unit 120 may receive input regarding actual pregnancy according to diagnosis results based on the ovulation test kits of other users among other users classified as the same as the user with the reliability, which is the probability of whether a state in question corresponds to an actual ovulation state according to the diagnosis result of the ovulation test kit of the user, and may calculate and provide the ratio of actual pregnancy among other users diagnosed with ovulation based on the input of the other users.

Alternatively, for example, the control unit 120 may provide 20% as the reliability of the diagnosis result of the ovulation test kit of the user indicative of actual ovulation when the probability that the diagnosis results of ovulation test kits are associated with the pregnancy of other users who are determined to be in an ovulation period is calculated as 20% in a group into which the user and other users with abdominal obesity are grouped based on the physical values of the user.

According to another embodiment, the control unit 120 may provide the reliability of the self-diagnosis apparatus to the user based on the reliability calculated based on the correlation between the self-diagnosis apparatuses used by other users included in the same group to which the user belongs.

For example, the control unit 120 may calculate the reliability of the ovulation test kits of a group to which the user belongs based on the diagnosis result of an ovulation test kit and the diagnosis result of a pregnancy test kit used by other obese users, and may provide the reliability of the ovulation test kit of the user based on the calculated reliability.

Meanwhile, the control unit 120 may identify the validity of the diagnosis result for the suitability for determination by additionally conducting a test on the reaction result of the self-diagnosis apparatus of the user according to the guide information of the self-diagnosis apparatus of the user.

To this end, the control unit 120 may identify whether the user has conducted the test according to the guide information of the self-diagnosis apparatus.

For example, the control unit 120 may identify whether the user has fasted according to the guide information before the test using the self-diagnosis apparatus through the checklist.

Alternatively, for example, the control unit 120 may identify whether the user has conducted a test using the self-diagnosis apparatus in a preset test cycle according to the guide information based on the user's analysis record for the diagnosis result of the self-diagnosis apparatus.

In addition, for example, the control unit 120 may obtain user input for each test process of the self-diagnosis apparatus, and may identify whether the user has conducted the test according to the test process based on whether the user input has been generated based on an elapsed period of time in each process.

The communication unit 130 may perform wired/wireless communication with another device or a network. To this end, the communication unit 130 may include a communication module configured to support at least one of various wired and wireless communication methods. For example, the communication module may be implemented in the form of a chipset.

The wireless communication supported by the communication unit 130 may be, for example, Wireless Fidelity (Wi-Fi), Wi-Fi Direct, Bluetooth, Ultra-Wide Band (UWB), or Near Field Communication (NFC) communication. In addition, the wired communication supported by the communication unit 130 may be, for example, USB or High Definition Multimedia Interface (HDMI) communication.

Various types of data such as files, applications, and programs may be installed and stored in the memory 140. The control unit 120 may access and use data stored in the memory 140, or may store new data in the memory 140.

Furthermore, the control unit 120 may execute a program installed in the memory 140.

The memory 140 may store guide information, which is information about a method of using the self-diagnosis apparatus.

For example, the memory 140 may store the name and barcode number of each self-diagnosis apparatus, and may store images acquired by photographing reaction results based on the diagnosis results of each self-diagnosis apparatus.

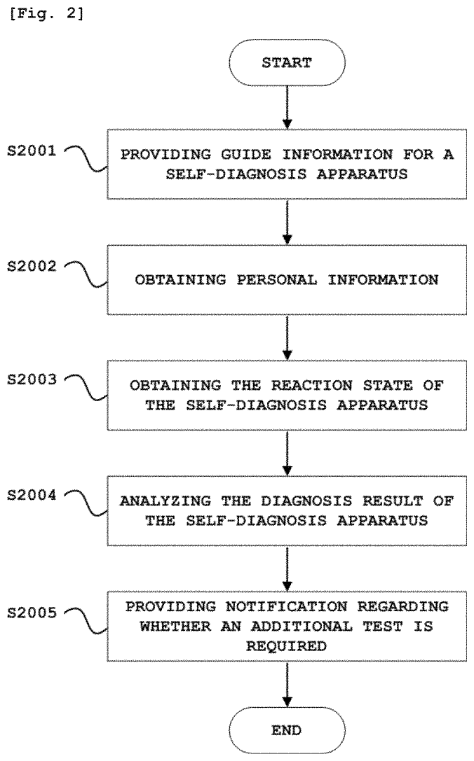

FIG. 2 is a flowchart illustrating a method of analyzing a self-diagnosis apparatus according to an embodiment.

The method of analyzing a self-diagnosis apparatus according to the embodiment shown in FIG. 2 includes steps that are performed in a time-series manner in the device 10 for analyzing a self-diagnosis apparatus shown FIG. 1. Accordingly, the descriptions that are omitted below but are given above in conjunction with the device 10 for analyzing a self-diagnosis apparatus shown FIG. 1 may also be applied to the method of analyzing a self-diagnosis apparatus according to the embodiment shown in FIG. 2.

First, the device 10 for analyzing a self-diagnosis apparatus may provide guide information, which is information about a method of using the self-diagnosis apparatus selected by a user, at step S2001.

For example, the self-diagnosis apparatus analyzing apparatus 10 may provide a list of at least one self-diagnosis apparatus to a user, and may receive a selection of a self-diagnosis apparatus to be used by the user.

Alternatively, for example, the device 10 for analyzing a self-diagnosis apparatus may photograph a barcode that is the identification information of the self-diagnosis apparatus possessed by the user, and may identify the self-diagnosis apparatus possessed by the user as an ‘ovulation test kit’ based on the photographed barcode.

In addition, the device 10 for analyzing a self-diagnosis apparatus may provide a checklist configured to obtain the user's personal information based on the selected self-diagnosis apparatus at step S2002.

For example, the device 10 for analyzing a self-diagnosis apparatus may provide a checklist that is an input window including queries on whether a preparation process for using the self-diagnosis apparatus has been performed, such as the storage state and expiration date of the self-diagnosis apparatus.

Alternatively, for example, the device 10 for analyzing a self-diagnosis apparatus may provide a checklist that is an input window including queries on the user's life-related queries, such as a medication being taken by the user, whether he or she has fasted, and the user's physical values.

In addition, the device 10 for analyzing a self-diagnosis apparatus may analyze the user's personal information obtained through the checklist, and may group the user and other users based on the analyzed user's life pattern.

For example, the device 10 for analyzing a self-diagnosis apparatus may obtain the analysis results in which the user's life cycle is irregular and the user has a life pattern of drinking a lot of water, and may group other users with a life pattern identical or similar to the user's life pattern by matching them.

In addition, the device 10 for analyzing a self-diagnosis apparatus may provide the guide information based on the analyzed life pattern.

For example, when the user has a life pattern of drinking a lot of water, the device 10 for analyzing a self-diagnosis apparatus may provide the user with guide information recommending the user to fast after 10 o'clock and go to bed before 10 o'clock.

Furthermore, for example, when the self-diagnosis apparatus selected at step S2001 is an ‘ovulation test kit,’ the device 10 for analyzing a self-diagnosis apparatus may provide a specimen collection method in which the user collects first urine after waking up and the lower 5 cm portion of the self-diagnosis apparatus is leached into the specimen for five seconds as the guide information. In addition, the device 10 for analyzing a self-diagnosis apparatus may receive a test start time input from the user, and may provide an alarm five seconds after the test start time.

In addition, the device 10 for analyzing a self-diagnosis apparatus may obtain a reaction state in which the self-diagnosis apparatus reacts with the specimen according to the user's test at step S2003.

Prior to this, according to an embodiment, the device 10 for analyzing a self-diagnosis apparatus may identify whether the user has conducted a test on the self-diagnosis apparatus according to the guide information at step S2002.

For example, the device 10 for analyzing a self-diagnosis apparatus may query whether the user has used the self-diagnosis apparatus according to the guide information, and may identify whether the test has been conducted based on the user's input.

In addition, the device 10 for analyzing a self-diagnosis apparatus may photograph the reaction state of the self-diagnosis apparatus.

For example, the device 10 for analyzing a self-diagnosis apparatus may obtain an input for the photographing of the self-diagnosis apparatus from the user, and may photograph the self-diagnosis apparatus.

Alternatively, for example, the device 10 for analyzing a self-diagnosis apparatus may perform photographing when a shape identical to that of the self-diagnosis apparatus is identified among the objects identified through a provided camera, or may perform photographing when a photographing location is identified based on the packaging on which a preset pattern is marked and the self-diagnosis apparatus is placed on the packaging.

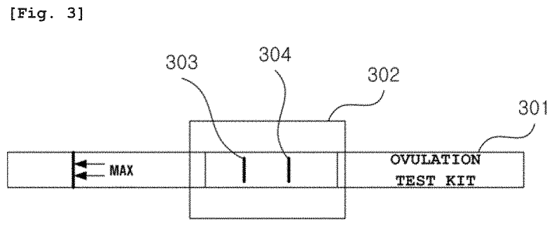

FIG. 3 is an exemplary view showing an image acquired by photographing the reaction result of a self-diagnosis apparatus. Referring to this drawing, the device 10 for analyzing a self-diagnosis apparatus may photograph a result check part 302 capable of showing the state of the reaction with a specimen in an ovulation test kit 301, which is a self-diagnosis apparatus, and may diagnose the ovulation state of a user based on a result line 303 indicative of the result of the reaction with the specimen and the control line 304 in the photographed result check part 302.

In addition, the device 10 for analyzing a self-diagnosis apparatus may analyze the diagnosis result of the self-diagnosis apparatus based on the stored reaction state of the self-diagnosis apparatus of another user at step S2004.

For example, the device 10 for analyzing a self-diagnosis apparatus may compare the result line attributable to the reaction with the specimen and the control line based on the image acquired by photographing the reaction state of the self-diagnosis apparatus. In this case, the device 10 for analyzing a self-diagnosis apparatus may analyze the diagnosis result based on the sharpness, color bleeding, and like of the result and control lines.

In this case, according to an embodiment, the device 10 for analyzing a self-diagnosis apparatus may analyze whether a result line or a control line is present and also analyze the sharpness of the result and control lines based on a method of reading the diagnosis result of the self-diagnosis apparatus.

For example, when the color of the result line spreads and becomes indistinguishable from the surroundings in the reaction state of a pregnancy test kit, which is a self-diagnosis apparatus, the device 10 for analyzing a self-diagnosis apparatus may compare the area and color of the area of the control line with those of the result line, and may provide the analysis result in which a diagnosis result is non-pregnancy when they are not the same.

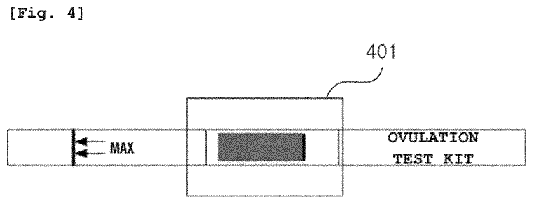

FIG. 4 is an exemplary view showing the reaction state of an ovulation test kit. Referring to this drawing, the device 10 for analyzing a self-diagnosis apparatus may provide the analysis result in which it is difficult to determine a diagnosis result when the test has not been conducted according to the guide information of the ovulation test kit, so that the color spreads to a portion of the result line 402 and thus it is difficult to check a result in the result check part 401 of the ovulation test kit.

Alternatively, according to another embodiment, the device 10 for analyzing a self-diagnosis apparatus may learn the relationship between the diagnosis result of another user and the reaction state of a self-diagnosis apparatus, and may analyze the user's diagnosis state based on the result of the learning.

For example, the device 10 for analyzing a self-diagnosis apparatus may learn a diagnosis result in which pregnancy is determined when a result line is present regardless of the sharpness of the result line in a pregnancy test kit, which is a self-diagnosis apparatus, and may diagnose pregnancy even when the color of the result line in the pregnancy test kit is light.

Alternatively, for example, the device 10 for analyzing a self-diagnosis apparatus may learn diagnosis results, such as imminent ovulation, before/after ovulation, and test failure, based on the sharpness of the result and control lines in an ovulation test kit, which is a self-diagnosis apparatus, and may accurately determine a diagnosis result according to the sharpness of each of the result and control lines of the ovulation test kit tested by the user.

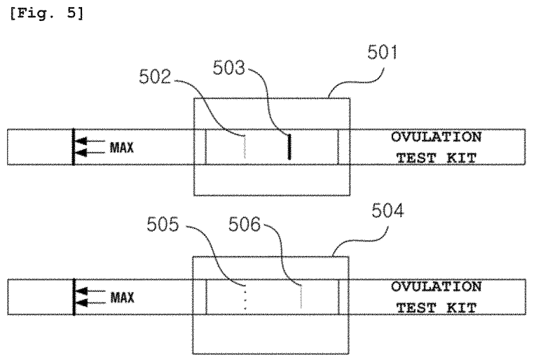

FIG. 5 is an exemplary view showing the reaction states of an ovulation test kit. Referring to this drawing, the device 10 for analyzing a self-diagnosis apparatus may learn the colors and thicknesses of the result and control lines 502 and 503 of the ovulation test kit 501 of another user diagnosed with imminent ovulation, and may compare each of the result and control lines 505 and 506 of the user's ovulation test device 504 with each of the learned result and control lines 502 and 503 of the ovulation test kit 501 of another user. In addition, the device 10 for analyzing a self-diagnosis apparatus may determine the diagnosis result to be ‘before/after ovulation’ because the sharpness of the result line 505 of the user is lower than that of the result line 502 of the other user even when the result line 505 of the user is present.

In addition, according to an embodiment, the device 10 for analyzing a self-diagnosis apparatus may compare the diagnosis result of another user belonging to the group into which the user is classified and the actual physical condition of the other user, and may analyze the reliability of the user's diagnosis result based on the probability of having the same result.

For example, when the probability of being diagnosed with actual ovulation is 40% even when the result line of the ovulation test kit of another user who is taking the same medication as the user is shape, the device 10 for analyzing a self-diagnosis apparatus may provide the analysis result in which the reliability of the diagnosis result in which ovulation is diagnosed because the result line of the ovulation test kit of the user is shape is 40%.

In addition, according to an embodiment, the device 10 for analyzing a self-diagnosis apparatus may analyze the validity of the diagnosis result using the self-diagnosis apparatus.

For example, the device 10 for analyzing a self-diagnosis apparatus may identify the time for which the self-diagnosis apparatus has been used by the user according to the guide information based on the analysis record of the diagnosis result of the self-diagnosis apparatus, and may analyze the validity of the diagnosis result by identifying whether the test has been conducted according to the guide information.

In addition, at step S2005, the device 10 for analyzing a self-diagnosis apparatus may notify the user of whether an additional test is required based on the diagnosis result analyzed at step S2004.

For example, the device 10 for analyzing a self-diagnosis apparatus may notify the user of an additional test when it is determined that the diagnosis result is unclear or the reaction state of the self-diagnosis apparatus is not valid.

Alternatively, for example, when tests are to be conducted at regular intervals, the device 10 for analyzing a self-diagnosis apparatus may provide notification that an additional test is to be conducted a preset period of time after the day on which the previous test is performed.

The term ‘unit’ used in the above-described embodiments means software or a hardware component such as a field-programmable gate array (FPGA) or application-specific integrated circuit (ASIC), and a ‘unit’ performs a specific role. However, a ‘unit’ is not limited to software or hardware. A ‘unit’ may be configured to be present in an addressable storage medium, and also may be configured to run one or more processors. Accordingly, as an example, a ‘unit’ includes components, such as software components, object-oriented software components, class components and task components, processes, functions, attributes, procedures, subroutines, segments in program code, drivers, firmware, microcode, circuits, data, a database, data structures, tables, arrays, and variables.

Each of the functions provided in components and ‘unit(s)’ may be coupled to a smaller number of components and ‘unit (s)’ or divided into a larger number of components and ‘unit(s).’

In addition, components and ‘unit(s)’ may be implemented to run one or more CPUs in a device or secure multimedia card.

The method of analyzing a self-diagnosis apparatus according to the embodiment described through FIG. 2 may be implemented in the form of a computer-readable medium that stores instructions and data that can be executed by a computer. In this case, the instructions and the data may be stored in the form of program code, and may generate a predetermined program module and perform a predetermined operation when executed by a processor. Furthermore, the computer-readable medium may be any type of available medium that can be accessed by a computer, and may include volatile, non-volatile, separable and non-separable media. Furthermore, the computer-readable medium may be a computer storage medium. The computer storage medium may include all volatile, non-volatile, separable and non-separable media that store information, such as computer-readable instructions, a data structure, a program module, or other data, and that are implemented using any method or technology. For example, the computer storage medium may be a magnetic storage medium such as an HDD, an SSD, or the like, an optical storage medium such as a CD, a DVD, a Blu-ray disk or the like, or memory included in a server that can be accessed over a network.

Furthermore, the method of analyzing a self-diagnosis apparatus according to the embodiment described through FIG. 2 may be implemented as a computer program (or a computer program product) including computer-executable instructions. The computer program includes programmable machine instructions that are processed by a processor, and may be implemented as a high-level programming language, an object-oriented programming language, an assembly language, a machine language, or the like. Furthermore, the computer program may be stored in a tangible computer-readable storage medium (for example, memory, a hard disk, a magnetic/optical medium, a solid-state drive (SSD), or the like).

Accordingly, the method of analyzing a self-diagnosis apparatus according to the embodiment described through FIG. 2 may be implemented in such a manner that the above-described computer program is executed by a computing device. The computing device may include at least some of a processor, memory, a storage device, a high-speed interface connected to memory and a high-speed expansion port, and a low-speed interface connected to a low-speed bus and a storage device. These individual components are connected using various buses, and may be mounted on a common motherboard or using another appropriate method.

In this case, the processor may process instructions within a computing device. An example of the instructions is instructions which are stored in memory or a storage device in order to display graphic information for providing a Graphic User Interface (GUI) onto an external input/output device, such as a display connected to a high-speed interface. As another embodiment, a plurality of processors and/or a plurality of buses may be appropriately used along with a plurality of pieces of memory. Furthermore, the processor may be implemented as a chipset composed of chips including a plurality of independent analog and/or digital processors.

Furthermore, the memory stores information within the computing device. As an example, the memory may include a volatile memory unit or a set of the volatile memory units. As another example, the memory may include a non-volatile memory unit or a set of the non-volatile memory units. Furthermore, the memory may be another type of computer-readable medium, such as a magnetic or optical disk.

In addition, the storage device may provide a large storage space to the computing device. The storage device may be a computer-readable medium, or may be a configuration including such a computer-readable medium. For example, the storage device may also include devices within a storage area network (SAN) or other elements, and may be a floppy disk device, a hard disk device, an optical disk device, a tape device, flash memory, or a similar semiconductor memory device or array.

The above-described embodiments are intended for illustrative purposes. It will be understood that those having ordinary knowledge in the art to which the present invention pertains can easily make modifications and variations without changing the technical spirit and essential features of the present invention. Therefore, the above-described embodiments are illustrative and are not limitative in all aspects. For example, each component described as being in a single form may be practiced in a distributed form. In the same manner, components described as being in a distributed form may be practiced in an integrated form.

The scope of protection pursued via the present specification should be defined by the attached claims, rather than the detailed description. All modifications and variations which can be derived from the meanings, scopes and equivalents of the claims should be construed as falling within the scope of the present invention.

1. A device for analyzing a diagnosis result of a self-diagnosis apparatus, the device comprising:
memory configured to store guide information, which is information about a method of using a self-diagnosis apparatus; and
a control unit configured to provide the guide information for the self-diagnosis apparatus selected by a user, and to analyze a diagnosis result of the self-diagnosis apparatus tested according to the provided guide information.

2. The device of claim 1, wherein the control unit provides a checklist configured to obtain the user's personal information.

3. The device of claim 2, wherein the control unit analyzes the user's life pattern based on the user's personal information, and provides the guide information based on the analyzed life pattern.

4. The device of claim 2, wherein the control unit classifies the user based on the user's personal information.

5. The device of claim 1, wherein the control unit photographs a reaction state in which the self-diagnosis apparatus reacts with a specimen according to the user's test, and analyzes the diagnosis result of the self-diagnosis apparatus based on a pre-stored reaction state of a self-diagnosis apparatus of another user.

6. The device of claim 5, wherein the control unit analyzes a reliability of the user's diagnosis result based on a diagnosis result of another user belonging to a group into which the user is classified.

7. The device of claim 1, wherein the control unit identifies whether the user uses the self-diagnosis apparatus according to the guide information based on an analysis record of the diagnosis result of the self-diagnosis apparatus.

8. A method of analyzing a self-diagnosis apparatus by which a device for analyzing a self-diagnosis apparatus analyzes a diagnosis result of a self-diagnosis apparatus, the method comprising:
providing guide information, which is information about a method of using a self-diagnosis apparatus selected by a user; and
analyzing a diagnosis result of the self-diagnosis apparatus tested according to the provided guide information.

9. The method of claim 8, further comprising providing a checklist configured to obtain the user's personal information.

10. The method of claim 9, further comprising:
analyzing the user's life pattern based on the user's personal information; and
providing the guide information based on the analyzed life pattern.

11. The method of claim 9, further comprising classifying the user based on the user's personal information.

12. The method of claim 8, wherein analyzing the diagnosis result of the self-diagnosis apparatus comprises;
photographing a reaction state in which the self-diagnosis apparatus reacts with a specimen according to the user's test; and
analyzing the diagnosis result of the self-diagnosis apparatus based on a pre-stored reaction state of a self-diagnosis apparatus of another user.

13. The method of claim 12, further comprising analyzing a reliability of the user's diagnosis result based on a diagnosis result of another user belonging to a group into which the user is classified.

14. The method of claim 8, further comprising identifying whether the user uses the self-diagnosis apparatus according to the guide information based on an analysis record of the diagnosis result of the self-diagnosis apparatus.

15. A computer-readable storage medium having stored thereon a program that performs the method set forth in claim 8.
